(19) United States
(12) Patent Application Publication
(10) Pub. No.: US 2008/0132554 A1
(43) Pub. Date: Jun. 5, 2008

(54) CRYSTAL FORM OF 2-(3-FLUORO-4-HYDROXYPHENYL)-7-VINYL-1,3-BENZOXAZOL-5-OL

(75) Inventors: Abdolsamad TADAYON, Kirkland (CA); Silvio IERA, Montreal (CA); Mahmoud MIRMEHRABI, Laval (CA)

Correspondence Address: Pepper Hamilton LLP/Wyeth, 400 Berwyn Park, 899 Cassatt Road, Berwyn, PA, 19312-1183, US

(73) Assignee: WYETH, Madison, NJ (US)

(21) Appl. No.: 11/943,076

(22) Filed: Nov. 20, 2007

Related U.S. Application Data
(60) Provisional application No. 60/860,253, filed on Nov. 21, 2006.

(51) Int. Cl. C07D 263/57; A61K 31/423; A61P 1/00; A61P 3/00; A61P 11/00; A61P 19/00; A61P 9/00; A61P 17/00; A61P 35/00

(52) U.S. Cl. 514/375; 548/224

(57) ABSTRACT

The present invention is directed to an anhydrate crystal form (Form D) of 2-(3-fluoro-4-hydroxyphenyl)-7-vinyl-1,3-benzoxazol-5-ol, an estrogenic receptor modulator useful in the treatment of, for example, diseases related to abnormal levels of estrogen.

This application claims benefit of priority to U.S. provisional patent application Ser. No. 60/860,253 filed Nov. 21, 2006, which is hereby incorporated by reference in its entirety.

### FIELD OF THE INVENTION

The present invention is directed to an anhydrate crystal form (designated as Form D herein) of 2-(3-fluoro-4-hydroxyphenyl)-7-vinyl-1,3-benzoxazol-5-ol, an estrogenic receptor modulator useful in the treatment of, for example, diseases related to abnormal levels of estrogen.

### BACKGROUND OF THE INVENTION

The pleiotropic effects of estrogens in mammalian tissues have been well documented, and it is now appreciated that estrogens affect many organ systems. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences. A class of “coregulatory” proteins can also interact with the ligand-bound receptor and further modulate its transcriptional activity. It has also been shown that estrogen receptors can suppress NF.kappa.B-mediated transcription in both a ligand-dependent and independent manner.

Accordingly, compounds which are estrogen receptor modulators are useful in the treatment or inhibition of conditions, disorders, or disease states that are at least partially mediated by an estrogen deficiency or excess, or which may be treated or inhibited through the use of an estrogenic agent. Such compounds can be particularly useful in treating a peri-menopausal, menopausal, or postmenopausal patient in which the levels of endogenous estrogens produced are greatly diminished. For example, estrogenic compounds are also useful in inhibiting or treating hot flushes, vaginal or vulvar atrophy, atrophic vaginitis, vaginal dryness, pruritus, dyspareunia, dysuria, frequent urination, urinary incontinence, and urinary tract infections. Other reproductive tract uses include the treatment or inhibition of dysfunctional uterine bleeding and endometriosis.

Certain substituted benzoxazole compounds have been found to be effective estrogenic receptor modulators. An example of such a benzoxazole is 2-(3-fluoro-4-hydroxyphenyl)-7-vinyl-1,3-benzoxazol-5-ol, shown below in Formula (I). The effectiveness of this compound as an estrogenic modulator, as well as its preparation, are reported in U.S. Pat. No. 6,794,403, which is hereby incorporated by reference in its entirety.

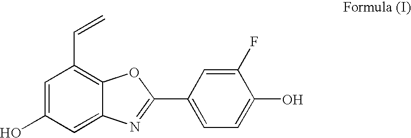

### 2-(3-fluoro-4-hydroxyphenyl)-7-vinyl-1,3-benzoxazol-5-ol

The crystal form of a particular drug (e.g., hydrate, solvate, polymorph, etc) is often an important determinant of the drug's ease of preparation, stability, solubility, storage stability, ease of formulation and in vivo pharmacology. Different crystal forms occur when a compound crystallizes in different lattice arrangements or where solvent molecules (including water molecules) are incorporated into the crystalline lattice, resulting in solids with different thermodynamic properties and stabilities specific to the particular form. It is entirely possible that one crystal form is preferable over another where certain aspects such as ease of preparation, stability, etc. are deemed to be critical. Similarly, greater solubility and/or superior pharmacokinetics may be the desired characteristics. An anhydrate crystal form and a monohydrate crystal form of the compound of Formula (I) have been reported in U.S. patent application Ser. No. 11/369,405, filed Mar. 06, 2006, which is hereby incorporated by reference in its entirety. Moreover, three additional different crystal forms of 2-(3-fluoro-4-hydroxyphenyl)-7-vinyl-1,3-benzoxazol-5-ol, two anhydrate crystal forms and another crystal form, have been disclosed in three U.S. provisional patent applications (U.S. provisional patent application Ser. No. 60/860,265 filed Nov. 21, 2006; U.S. provisional patent application Ser. No. 60/860,246 filed Nov. 21, 2006; and U.S. provisional patent application Ser. No. 60/860,248 filed Nov. 21, 2006), each of which is incorporated by reference herein in its entirety.

Because improved drug formulations showing, for example, better bioavailability or better stability are consistently sought, there is an ongoing need for new or purer crystal forms of existing drug molecules. The crystal form (Form D) of 2-(3-fluoro-4-hydroxyphenyl)-7-vinyl-1,3-benzoxazol-5-ol described herein are directed toward this and other important ends.

### SUMMARY OF THE INVENTION

The present invention provides an anhydrate crystal form (designated as Form D herein) of 2-(3-fluoro-4-hydroxyphenyl)-7-vinyl-1,3-benzoxazol-5-ol, characterized according to the powder X-ray diffraction data, differential scanning calorimetry, and thermogravimetric analysis provided herein.

The present invention further provides compositions containing the anhydrate crystal form (Form D) of the invention.

The present invention further provides a method of preparing the anhydrate crystal form (Form D) of the invention comprising precipitating a solid from a solution which comprises 2-(3-fluoro-4-hydroxyphenyl)-7-vinyl-1,3-benzoxazol-5-ol and an organic solvent, and drying the solid from the precipitation.

The present invention further provides compounds (the crystal form: Form D) prepared by the above methods.

The present invention further provides methods of modulating an estrogen receptor comprising contacting the receptor with the crystal form (Form D) of the invention.

The present invention further provides methods of treating prostatitis, interstitial cystitis, inflammatory bowel disease, Crohn's disease, ulcerative proctitis, colitis, prostatic hypertrophy, uterine leiomyomas, breast cancer, endometrial cancer, polycystic ovary syndrome, endometrial polyps, endometriosis, benign breast disease, adenomyosis, ovarian cancer, melanoma, prostrate cancer, colon cancer, glioma, astioblastomia, free radical induced disease states, vaginal or vulvar atrophy, atrophic vaginitis, vaginal dryness, pruritus, dyspareunia, dysuria, frequent urination, urinary incontinence, urinary tract infections, vasomotor symptoms, arthritis, joint swelling or erosion, joint damage secondary to arthroscopic or surgical procedures, psoriasis, dermatitis, ischemia, reperfusion injury, asthma, pleurisy, multiple sclerosis, systemic lupus erythematosis, uveitis, sepsis, hemmorhagic shock, or type II diabetes, in a mammal in need thereof, which comprises providing to the mammal a therapeutically effective amount of the crystal form (Form D) of the invention.

The present invention further provides methods of lowering cholesterol, triglycerides, Lp(a), or LDL levels; inhibiting or treating hypercholesteremia, hyperlipidemia, cardiovascular disease, atherosclerosis, hypertension, peripheral vascular disease, restenosis, or vasospasm; or inhibiting vascular wall damage from cellular events leading toward immune mediated vascular damage in a mammal in need thereof, which comprises providing to the mammal a therapeutically effective amount of the crystal form (Form D) of the invention.

The present invention further provides methods of providing cognition enhancement or neuroprotection; or treating or inhibiting senile dementias, Alzheimer's disease, cognitive decline, stroke, anxiety, or neurodegenerative disorders in a mammal in need thereof, which comprises providing to the mammal an effective amount of the crystal form (Form D) of the invention.

The present invention further provides methods of inhibiting conception in a mammal in need thereof, which comprises providing to the mammal an effective amount of the crystal form (Form D) of the invention.

### BRIEF DESCRIPTION OF THE DRAWINGS

FIG. 1 depicts X-Ray powder diffraction (XRPD) pattern for the anhydrate crystal form (Form D) of the invention.

FIG. 2 depicts a differential scanning calorimetry (DSC) thermogram of the anhydrate crystal form (Form D) of the invention.

FIG. 3 depicts a thermogravimetric analysis (TGA) of the anhydrate crystal form (Form D) of the invention.

### DETAILED DESCRIPTION

### Anhydrate Crystal Form

The present invention provides, inter alia, an anhydrate crystal form (designated as Form D herein) of 2-(3-fluoro-4-hydroxyphenyl)-7-vinyl-1,3-benzoxazol-5-ol, the compound of Formula (I). The crystal form (Form D) of the compound of Formula (I) can be identified by its unique solid state signatures with respect to, for example, X-ray powder diffraction (XRPD) and other solid state methods. Further characterization with respect to water or solvent content of the crystal forms can be gauged by any of various routine methods such as thermogravimetric analysis (TGA) and DSC and other techniques. For DSC, it is known that the temperatures observed will depend upon the rate of temperature change as well as sample preparation technique and the particular instrument employed. Thus, the values reported herein relating to DSC thermograms can vary by plus or minus about 4° C. Accordingly, the term “about” as used in connection with a given DSC temperature value, is intended to mean plus or minus 4° C. For XRPD, the relative intensities of the peaks can vary, depending upon the sample preparation technique, the sample mounting procedure and the particular instrument employed. Moreover, instrument variation and other factors can often affect the 2-theta values. Therefore, the peak assignments of diffraction patterns can vary by plus or minus about 0.2°. Accordingly, the term “about” as used in connection with a given 2-theta value, is intended to mean plus or minus 0.2°.

The physical properties and X-ray data distinguishing the crystal form (Form D) of the invention are summarized in Tables 1 and 2 below.

TABLE 1
Physical properties of the anhydrate crystal form of Form D

| | |
|---|---|
| TGA | less than about 0.7% |
| DSC | Exotherm onset ~102° C.; and melt onset ~246° C. |
| XRPD | 6.1°, 10.6°, and 16.2° 2θ |

TABLE 2
X-ray data of the anhydrate crystal form of Form D

| Peak position, 2θ° | Peak Description |
|---|---|
| 6.1 | VS |
| 9.6 | W |
| 10.6 | Strongest |
| 12.3 | W |
| 14.6 | W |
| 16.2 | M |

VS: very high peak intensity
M: middle range peak intensity
W: relatively weak peak intensity

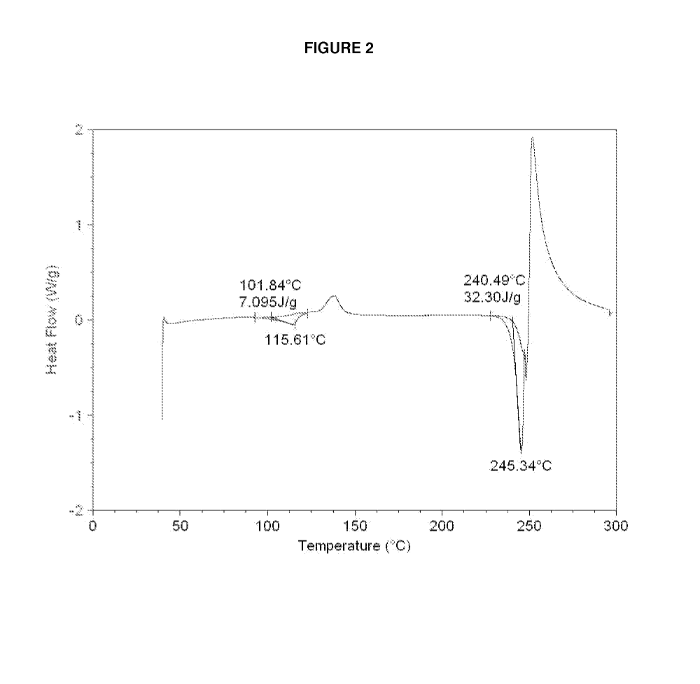

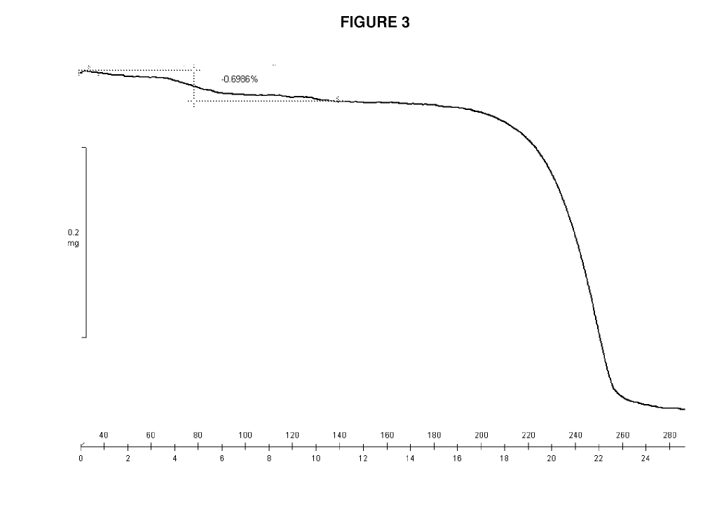

As can be seen from Table 1, the anhydrate (anhydrous) crystal form (Form D) contains little water content, showing less than about 0.7% by TGA (see also FIG. 3) and a lack of a dehydration endotherm in the DSC (see also FIG. 2).

In accordance with the distinguishing features provided by DSC and TGA analysis, the present invention provides an anhydrate (anhydrous) crystal form (Form D) of the compound of Formula (I) having a differential scanning calorimetry trace comprising an exotherm having an onset at about 102° C., a melting endotherm having an onset at about 241° C. and substantially lacking an endotherm corresponding to a dehydration event. In some embodiments, the crystal form has a differential scanning calorimetry trace substantially as shown in FIG. 2. In some embodiments, the crystal form can have a thermogravimetric analysis profile showing less than about 1.5%, about 1.2%, about 1%, less than about 0.8%, less than about 0.7%, less than about 0.6%, less than about 0.5%, less than about 0.3%, less than about 0.2%, or less than about 0.1% weight loss from about 60 to about 150° C. In further embodiments, the crystal form can have a thermogravimetric analysis profile showing less than about 1.2%, about 1%, less than about 0.8%, less than about 0.7%, less than about 0.6%, or less than about 0.5% weight loss from about 60 to about 150° C. In yet further embodiments, the crystal form can have a have a thermogravimetric analysis profile substantially as shown in FIG. 3.

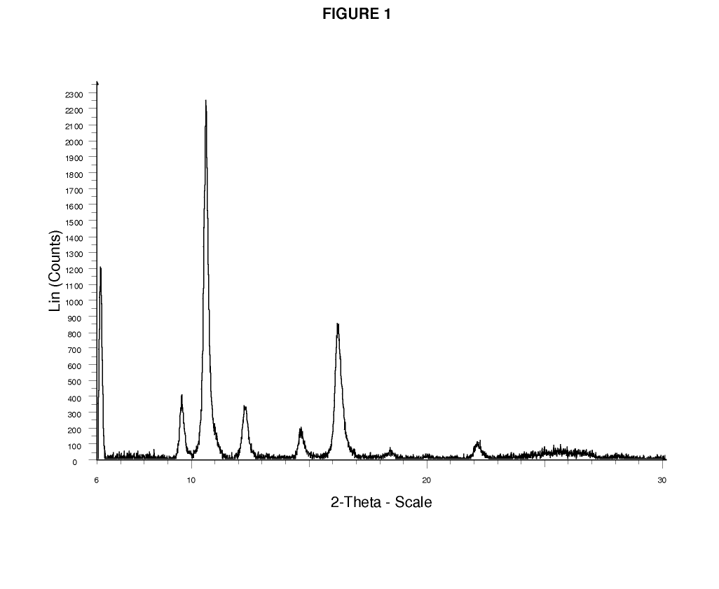

The crystal form (Form D) has a distinct XRPD pattern (see, e.g., FIG. 1), allowing characterization thereof based on the unique spectral signature. Accordingly, the present invention provides an anhydrous crystal form of the compounds of Formula I having an X-ray powder diffraction pattern comprising peaks, in terms of 2θ, at about 6.1°, about 10.6°, and about 16.2°. In some further embodiments, the crystal form has an X-ray powder diffraction pattern further comprising at least one peak, in terms of 2θ, selected from at about 9.6°, about 12.3°, and about 14.6°. In some embodiments, the crystal form has an X-ray powder diffraction pattern comprising peaks, in terms of 2θ, at about 6.1°, about 9.6°, about 10.6°, and about 16.2°. In some embodiments, the crystal form has an X-ray powder diffraction pattern comprising peaks, in terms of 2θ, at about 6.1°, about 10.6°, about 12.3°, and about 16.2°. In some embodiments, the crystal form has an X-ray powder diffraction pattern comprising peaks, in terms of 2θ, at about 6.1°, about 9.6°, about 10.6°, about 12.3°, about 14.6°, and about 16.2°. In further embodiments, the crystal form has an X-ray powder diffraction pattern substantially as shown in FIG. 1.

### Compositions

The present invention further provides compositions containing the crystal form (Form D) of the invention. In some embodiments, the compositions of the invention include at least about 50%, at least about 60%, at least about 70%, at least about 80%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, by weight of the anhydrate crystal form of the compound of Formula (I). In some embodiments, the compositions of the invention contain a mixture of the anhydrate crystal form and other crystal forms or amorphous forms of the compound of Formula (I). In some embodiments, compositions of the invention include the anhydrate crystal form and a pharmaceutically acceptable carrier. In some embodiments, the compositions further include an additional active ingredient such as a progestin.

### Preparations

The anhydrate crystal form (Form D) of the invention can be prepared by any of various suitable means. For example, the anhydrate can be prepared by precipitating a solid from a solution which comprises 2-(3-fluoro-4-hydroxyphenyl)-7-vinyl-1,3-benzoxazol-5-ol and an organic solvent, and drying the solid from the precipitation. In some embodiments, the solution is prepared in a suitable organic solvent near saturation. Suitable organic solvents for precipitating the anhydrate crystal form include polar organic solvents such as ketones (e.g., acetone or the like), alcohols (e.g., methanol, ethanol and isopropanol), organic nitrile (e.g., acetonitrile or the like), and mixture thereof. Suitable polar organic solvents for precipitating the anhydrate crystal form include water-miscible organic solvents (i.e., those miscible with water). Non-limiting example water miscible organic solvents include ketones (e.g., acetone or the like), alcohols (e.g., methanol, ethanol and isopropanol), organic nitrile (e.g., acetonitrile or the like), and mixture thereof. In some embodiments, the anhydrate is precipitated from a solvent containing an alcohol, a ketone or an organic nitrile. In some embodiments, the anhydrate is precipitated from a solvent containing an alcohol or a ketone. In some embodiments, the anhydrate is precipitated from a solvent containing methanol, ethanol, isopropanol, acetone or acetonitrile. In some embodiments, the anhydrate is precipitated from a solvent containing methanol, ethanol, isopropanol or acetone.

Precipitation of the solid can be induced by any of the various well known methods of precipitation including adding antisolvent to, or cooling the solution, or combination thereof. For example, the solid can be precipitated by addition of antisolvents to a solution in which the compound of Formula (I) is dissolved. An antisolvent is a solvent in which the compound of Formula (I) or a solvate thereof (including a hydrate thereof) is poorly soluble. Suitable antisolvents comprise water. In some embodiments, water is used as the antisolvent for the precipitation. The relative volume of the antisovlent to the solution is greater than about 1, from about 1.5 to about 3.5, or from about 2 to about 3. In some embodiments, the precipitation of the solid from the solution (comprising compound of Formula I) can be induced by fast addition of the antisolvent.

As used herein, “fast addition” of the antisolvent refers to adding the antisolvent to the solution (comprising compound of Formula I) in less than about 20 minutes, about 15 minutes, about 10 minutes, about 5 minutes, about 2 minutes, or about 1 minute. In some embodiments, although the fast addition is intended to be immediate, it can be delayed by the experimental conditions such as equipment setup. In some embodiments, the fast addition is carried out at a temperature of about 10° C. to about 70° C., about 20° C. to about 50° C., or about 20° C. to about 30° C. (room temperature). In some embodiments, before the addition, both the antisolvent and the solution are heated to a at a temperature of about 10° C. to about 70° C., about 20° C. to about 50° C., or about 20° C. to about 30° C. (thus maintaining the temperature during the addition, so as to avoid precipitation caused by cooling). In some embodiments, the “fast addition” of the antisolvent to the solution is carried out by adding the solution to the antisolvent.

After the fast addition of the antisolvent, precipitation occurs and a suspension forms. The suspension can optionally be stirred for an additional period of time, for example less than about 1 hour, about 30 minutes, about 5 minutes, or about 2 minutes. In some embodiments, no additional stirring is carried out. The solid is then collected, for example by filtering the suspension, and the solid obtained is dried optionally under reduced pressure (e.g., in a vacuum oven at 5-10 mmHg) and optionally at an elevated temperature (i.e., greater than room temperature), e.g., at about 50° C., to afford the crystal form of the present invention. In some embodiments, the solid is dried under reduced pressure (e.g., in a vacuum oven) and at an elevated temperature (e.g., at about 50° C.) to afford the crystal form. The final product obtained can have a purity of at least about 50%, at least about 60%, at least about 70%, at least about 80%, at least about 90%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, by weight of the anhydrate crystal form of the present invention. In some embodiments, because of the water in the antisolvent, the product may further contain a hydrate crystal form of the compound of Formula (I) such as the one disclosed in U.S. patent application Ser. No. 11/369,405, filed Mar. 06, 2006, which is hereby incorporated by reference in its entirety.

Not wishing to be bound by any particular theory, it is postulated that the solid precipitated from the solution comprises a hydrate of the compound of Formula (I), and that dehydration of the hydrate occurs during the drying process to provide the anhydrate crystal form of the present invention.

The anhydrate crystal form (Form D) can be converted to other crystal forms under suitable conditions. For example, after the anhydrate crystal form is suspended and stirred in water at room temperature for seven days, most of the anhydrate crystal form is converted to a hydrate crystal form (the hydrate crystal form has been disclosed in U.S. patent application Ser. No. 11/369,405, filed Mar. 06, 2006). For another example, after the anhydrate crystal form is suspended and stirred in methanol at room temperature for seven days, another anhydrate crystal form is obtained (the other anhydrate crystal form has been disclosed in U.S. provisional patent application Ser. No. 60/860,246 filed Nov. 21, 2006, which is hereby incorporated by reference in its entirety). For yet another example, after the anhydrate crystal form is suspended and stirred in ethanol or acetone at room temperature for seven days, another anhydrate crystal form is obtained (the other anhydrate crystal form has been disclosed in U.S. patent application Ser. No. 11/369,405, filed Mar. 06, 2006, which is hereby incorporated by reference in its entirety).

### Methods of Use and Pharmaceutical Formulations

The crystal form (Form D) of this invention is an estrogen receptor modulator useful in the treatment or inhibition of conditions, disorders, or disease states that are at least partially mediated by an estrogen deficiency or excess, or which can be treated or inhibited through the use of an estrogenic agent. Accordingly, the present invention provides a method of modulating an estrogen receptor comprising contacting the receptor with a crystal form of the invention. The crystal form (Form D) of this invention is particularly useful in treating a peri-menopausal, menopausal, or postmenopausal patient in which the levels of endogenous estrogens produced are greatly diminished. Menopause is generally defined as the last natural menstrual period and is characterized by the cessation of ovarian function, leading to the substantial diminution of circulating estrogen in the bloodstream. As used herein, menopause also includes conditions of decreased estrogen production that may be surgically, chemically, or be caused by a disease state which leads to premature diminution or cessation of ovarian function.

The crystal form (Form D) of this invention is also useful in inhibiting or treating other effects of estrogen deprivation including, hot flushes, vaginal or vulvar atrophy, atrophic vaginitis, vaginal dryness, pruritus, dyspareunia, dysuria, frequent urination, urinary incontinence, and urinary tract infections. Other reproductive tract uses include the treatment or inhibition of dysfunctional uterine bleeding. The crystal form is also useful in treating or inhibiting endometriosis.

The crystal form (Form D) of this invention is also active in the brain and are therefore useful for inhibiting or treating Alzheimer's disease, cognitive decline, decreased libido, senile dementia, neurodegenerative disorders, depression, anxiety, insomnia, schizophrenia, and infertility. The crystal form (Form D) of this invention is also useful in treating or inhibiting benign or malignant abnormal tissue growth including, glomerulosclerosis, prostatic hypertrophy, uterine leiomyomas, breast cancer, scleroderma, fibromatosis, endometrial cancer, polycystic ovary syndrome, endometrial polyps, benign breast disease, adenomyosis, ovarian cancer, melanoma, prostate cancer, cancers of the colon, and CNS cancers, such as glioma or astioblastomia.

The crystal form (Form D) of this invention is cardioprotective and is antioxidants, and is useful in lowering cholesterol, triglycerides, Lp(a), and LDL levels; inhibiting or treating hypercholesteremia, hyperlipidemia, cardiovascular disease, atherosclerosis, peripheral vascular disease, restenosis, and vasospasm; and inhibiting vascular wall damage from cellular events leading toward immune mediated vascular damage. The compound of this invention is also useful in treating disorders associated with inflammation or autoimmune diseases, including inflammatory bowel disease (Crohn's disease, ulcerative colitis, indeterminate colitis), arthritis (rheumatoid arthritis, spondyloarthropathies, osteoarthritis), pleurisy, ischemia/reperfusion injury (e.g. stroke, transplant rejection, myocardial infarction, etc.), asthma, giant cell arteritis, prostatitis, uveitis, psoriasis, multiple sclerosis, systemic lupus erythematosus and sepsis.

The crystal form (Form D) of this invention is also useful in treating or inhibiting ocular disorders, including cataracts, uveitis, and macular degeneration, and in treating skin conditions such as aging, alopecia, and acne.

The crystal form (Form D) of this invention is also useful in treating or inhibiting metabolic disorders such as type-II diabetes, of lipid metabolism, and of appetite (e.g. anorexia nervosa and bulimia).

The crystal form (Form D) in this invention is also useful in treating or inhibiting bleeding disorders such as hereditary hemorrhagic telangiectasia, dysfunctional uterine bleeding, and combating hemorrhagic shock.

The crystal form (Form D) of this invention is useful in disease states where amenorrhea is advantageous, such as leukemia, endometrial ablations, chronic renal or hepatic disease or coagulation diseases or disorders.

The crystal form (Form D) of this invention can be used as a contraceptive agent, particularly when combined with a progestin.

Methods of treating the diseases and syndromes listed herein are understood to involve administering to an individual in need of such treatment a therapeutically effective amount of a crystal form of the invention, or composition containing the same. As used herein, the term “treating” in reference to a disease is meant to refer to preventing, inhibiting and/or ameliorating the disease.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

As used herein, the term “contacting” refers to the bringing together of indicated moieties in an in vitro system or an in vivo system. For example, “contacting” an estrogen receptor with a crystal form of the invention includes the administration of a crystal form of the present invention to an individual or patient, such as a human, having an estrogen receptor, as well as, for example, introducing a crystal form of the invention into a sample containing a cellular or purified preparation containing the estrogen receptor.

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. Effective administration of the crystal forms of this invention may be given at an oral dose of from about 0.1 mg/day to about 1,000 mg/day. Preferably, administration will be from about 10 mg/day to about 600 mg/day, more preferably from about 50 mg/day to about 600 mg/day, in a single dose or in two or more divided doses. The projected daily dosages are expected to vary with route of administration.

Such doses may be administered in any manner useful in directing the active compounds herein to the recipient's bloodstream, including orally, via implants, parentally (including intravenous, intraperitoneal, intraarticularly and subcutaneous injections), rectally, intranasally, topically, ocularly (via eye drops), vaginally, and transdermally.

Oral formulations containing the active crystal forms of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the crystal form (Form D) directly to the airways in the form of an aerosol.

The crystal form (Form D) of this invention may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

In order that the invention disclosed herein may be more efficiently understood, examples are provided below. It should be understood that these examples are for illustrative purposes only and are not to be construed as limiting the invention in any manner.

### EXAMPLES

### Example 1

### Preparation of the Anhydrate Crystal Form: Form D (Preparation 1)

Solid 2-(3-fluoro-4-hydroxyphenyl)-7-vinyl-1,3-benzoxazol-5-ol (0.2 g) was dissolved in acetone (2 ml) at about 50° C. The suspension was filtered. To the solution, with stirring, was added water (6 ml) in one portion. The solid formed was filtered off and dried at 45-55° C., 5-10 mm Hg, to give the targeted crystalline form (Form D). Fast addition of the filtrate solution to water followed by drying at 45-55° C., 5-10 mm Hg, yielded the same crystalline form (Form D).

### Example 2

### Preparation of the Anhydrate Crystal Form: Form D (Preparation 2)

Solid 2-(3-fluoro-4-hydroxyphenyl)-7-vinyl-1,3-benzoxazol-5-ol (1 g) was added to methanol (3 ml) at 50° C. and stirred for 24 hrs. The resulting suspension was filtered. To the filtrate, with stirring, was added water at room temperature (6 ml) in one portion. The solid formed was filtered off and dried at 45-55° C., 5-10 mm Hg, to give the targeted crystalline form (Form D). Fast addition of the filtrate solution to water, followed by drying at 45-55° C., 5-10 mm Hg, yielded the same crystalline form (Form D).

### Example 3

### Preparation of the Anhydrate Crystal Form: Form D (Preparation 3)

Solid 2-(3-fluoro-4-hydroxyphenyl)-7-vinyl-1,3-benzoxazol-5-ol (0.36 g) was dissolved in ethanol (2 ml) at 70° C. To the solution, with stirring, was added water (at room temperature 6 ml) in one portion. The solid formed was filtered off and dried at 45-55° C., 5-10 mm Hg, to give the targeted crystalline form (Form D). Fast addition of the filtrate solution to water, followed by drying at 45-55° C., 5-10 mm Hg, yielded the same crystalline form (Form D).

### Example 4

### Preparation of the Anhydrate Crystal Form: Form D (Preparation 4)

Solid 2-(3-fluoro-4-hydroxyphenyl)-7-vinyl-1,3-benzoxazol-5-ol (0.28 g) was dissolved in isopropanol (2 ml) at 70° C. To the solution, with stirring, was added water (6 ml) in one portion. The solid formed was filtered off and dried at 45-55° C., 5-10 mm Hg, to give the targeted crystalline form (Form D). Fast addition of the filtrate solution to water, followed by drying at 45-55° C., 5-10 mm Hg, yielded the same crystalline form (Form D).

### Example 5

### Preparation of the Anhydrate Crystal Form: Form D (Preparation 5)

Solid 2-(3-fluoro-4-hydroxyphenyl)-7-vinyl-1,3-benzoxazol-5-ol (1 g) was added in acetonitrile (3 ml) at 70° C. and stirred for 24 hrs. The suspension was filtered. To the filtrate, with stirring, was added water (8 ml) in one portion. The solid formed was filtered off and dried at 45-55° C., 5-10 mm Hg. Alternatively, the solid was formed by fast addition of the filtrate solution to water, and the solid was dried at 45-55° C., 5-10 mm Hg, The solid formed (and dried) was a mixture of the crystalline form discussed herein (Form D) and the hydrate crystal form of the compound of Formula (I) disclosed in U.S. patent application Ser. No. 11/369,405, filed Mar. 06, 2006.

### Example 6

### Acquisition of X-Ray Powder Diffraction Data of Form D

X-Ray data (e.g., see FIG. 1 and Table 2) was acquired using an X-ray powder diffractometer (Bruker-axs, model D8 advance) having the following parameters: voltage 40 kV, current 40.0 mA, scan range (2θ) 6 to 30°, scan step size 0.01°, total scan time 29 minutes, with a Ni filter, detector slit 0.2 mm, and antiscattering slit 1 mm.

### Example 7

### Acquisition of Differential Scanning Calorimetry Data of Form D

Differential scanning calorimetry data (see FIG. 2) was collected using a DSC (TA instrument, model Q1000) under the following parameters: 50 mL/min purge gas (N₂), scan range 37 to 300° C., scan rate 10° C./min.

### Example 8

### Acquisition of Thermogravimetric Analysis Data of Form D

Thermogravimetric analysis data (see FIG. 3) was collected using a TGA instrument (Mettler Toledo, model TGA/SDTA 851e) under the following parameters: 40 mL/min purge gas (N₂); scan range 30 to 300° C., scan rate 20° C./min.

### Example 9

### Preparation of a Pharmaceutical Formulation and Composition Containing The Anhydrate Crystal Form (Form D) of the Invention (Unit Dose of 75 mg/Tablet)

The pharmaceutical formulation is prepared by steps 1-7 of the following procedure utilizing the weight/weight percentages (% wt/wt) of the ingredients shown in the table below. The tablets are prepared by steps 8-10 of the following procedure. Each tablet contained the unit dose amounts shown in the table below.

### 1. An aqueous solution of polyvinylpyrrolidone (povidone K25) and sodium lauryl sulfate in purified water is prepared.

### 2. The anhydrate crystal form (Form D) of 2-(3-fluoro-4-hydroxyphenyl)-7-vinyl-1,3-benzoxazol-5-ol is mixed with a portion of mannitol (Pearlitol 200SD), and then the mixture is passed through an appropriate screen and placed in a high shear mixer bowl.

### 3. The remainder of the mannitol, microcrystalline cellulose (Avicel pH 113), and croscarmellose sodium is passed through an appropriate screen into the mixer bowl and mixed.

### 4. The blend from Step 3 is granulated using the Step 1 solution, and is followed with additional purified water if needed.

### 5. The Step 4 granulation is dried and passed through an appropriate screen.

### 6. The magnesium stearate is passed through an appropriate screen.

### 7. The magnesium stearate is premixed with an equal portion of the blend in Step 5, and then the premix is added to the remainder of the Step 5 material and mixed in a blender.

### 8. The final blend from Step 7 is compressed into tablets using a suitable tablet press.

### 9. A 7.5% solid solution of Opaglos 2 is prepared.

### 10. A sufficient amount of coating solution is applied on the tablets to provide a 3.0% wt/wt increase in dried tablet weight.

Composition of the Pharmaceutical Formulation and Tablet

| | % wt/wt in the | |
|---|---|---|
| | pharmaceutical | Unit Dose |
| Ingredient | formulation batch | (mg/tablet) |
| The Anhydrate Crystal Form (Form | 25.0 | 75.0 |
| D) of 2-(3-fluoro-4-hydroxyphenyl)-7- | | |
| vinyl-1,3-benzoxazol-5-ol | | |
| Mannitol (Pearlitol 200SD) ^{a} | 51.5 | 154.5 |
| Microystalline Cellulose (Avicel PH | 15.0 | 45.0 |
| 113) | | |
| Croscarmellose Sodium | 4.0 | 12.0 |
| Polyvinylpyrrolidone (Povidone K25) | 2.0 | 6.0 |
| Sodium Lauryl Sulfate | 2.0 | 6.0 |
| Magnesium Stearate | 0.5 | 1.5 |
| Purified Water ^{b} | — | — |
| Total | 100.0% | 300.0 |
| Film Coat | 3.0 | 9.0 |
| Opaglos 2, green | | |
| 97W11753 | | |

^{a} If assay is other than 100.0%, adjust the amount of input against mannitol accordingly.
^{b} Used in the process, but does not appear in the final tablet product.

### Example 10

### Preparation of a Pharmaceutical Formulation and Composition Containing the Anhydrate Crystal Form (Form D) of the Invention (Tablet Unit Dose of 25 mg/Tablet)

The pharmaceutical formulation is prepared by steps 1-7 of the procedure of Example 9, utilizing the weight/weight percentages (% wt/wt) of the ingredients shown in the table below. The tablets are prepared by steps 8-10 of the procedure of Example 9. Each tablet contained the unit dose amounts shown in the table below.

Composition of the Tablet:

| | % wt/wt in the | |
|---|---|---|
| | pharmaceutical | Unit Dose |
| Ingredient | formulation batch | (mg/tablet) |
| The Anhydrate Crystal Form (Form | 25.0 | 25.0 |
| D) of 2-(3-fluoro-4-hydroxyphenyl)-7- | | |
| vinyl-1,3-benzoxazol-5-ol | | |
| Mannitol (Pearlitol 200SD) ^{a} | 51.5 | 51.5 |
| Microystalline Cellulose (Avicel PH | 15.0 | 15.0 |
| 113) | | |
| Croscarmellose Sodium | 4.0 | 4.0 |
| Polyvinylpyrrolidone (Povidone K25) | 2.0 | 2.0 |
| Sodium Lauryl Sulfate | 2.0 | 2.0 |
| Magnesium Stearate | 0.5 | 0.5 |
| Purified Water ^{b} | — | — |
| Total | 100.0% | 100.0 |
| Film Coat | 3.0 | 3.0 |
| Opaglos 2, green | | |
| 97W11753 | | |

^{a} If assay is other than 100.0%, adjust the amount of input against mannitol accordingly.
^{b} Used in the process, but does not appear in the final tablet product.

### Example 11

### Preparation of a Pharmaceutical Formulation and Composition Containing the Anhydrate Crystal Form (Form D) of the Invention (Tablet Unit Dose of 5 mg/Tablet)

The pharmaceutical formulation is prepared by steps 1-7 of the procedure of Example 9, utilizing the weight/weight percentages (% wt/wt) of the ingredients shown in the table below. The tablets are prepared by steps 8-10 of the procedure of Example 9. Each tablet contained the unit dose amounts shown in the table below.

Composition of the Tablet:

| | % wt/wt in the | |
|---|---|---|
| | pharmaceutical | Unit Dose |
| Ingredient | formulation batch | (mg/tablet) |
| The Anhydrate Crystal Form (Form | 5.0 | 5.0 |
| D) of 2-(3-fluoro-4-hydroxyphenyl)-7- | | |
| vinyl-1,3-benzoxazol-5-ol | | |
| Mannitol (Pearlitol 200SD) ^{a} | 71.5 | 71.5 |
| Microystalline Cellulose (Avicel PH | 15.0 | 15.0 |
| 113) | | |
| Croscarmellose Sodium | 4.0 | 4.0 |
| Polyvinylpyrrolidone (Povidone K25) | 2.0 | 2.0 |
| Sodium Lauryl Sulfate | 2.0 | 2.0 |
| Magnesium Stearate | 0.5 | 0.5 |
| Purified Water ^{b} | — | — |
| Total | 100.0% | 300.0 |
| Film Coat | 3.0 | 3.0 |
| Opaglos 2, green | | |
| 97W11753 | | |

^{a} If assay is other than 100.0%, adjust the amount of input against mannitol accordingly.
^{b} Used in the process, but does not appear in the final tablet product.

### Example 12

### Preparation of a Pharmaceutical Formulation and Composition Containing the Anhydrate Crystal Form (Form D) of the Invention (Tablet Unit Dose of 150 mg/Tablet)

The pharmaceutical formulation is prepared by steps 1-7 of the procedure of Example 9, utilizing the weight/weight percentages (% wt/wt) of the ingredients shown in the table below. The tablets are prepared by steps 8-10 of the procedure of Example 9. Each tablet contained the unit dose amounts shown in the table below.

Composition of the Tablet:

| | % wt/wt in the | |
|---|---|---|
| | pharmaceutical | Unit Dose |
| Ingredient | formulation batch | (mg/tablet) |
| The Anhydrate Crystal Form (Form | 25.0 | 150.0 |
| D) of 2-(3-fluoro-4-hydroxyphenyl)-7- | | |
| vinyl-1,3-benzoxazol-5-ol | | |
| Mannitol (Pearlitol 200SD) ^{a} | 51.5 | 309.0 |
| Microystalline Cellulose (Avicel PH | 15.0 | 90.0 |
| 113) | | |
| Croscarmellose Sodium | 4.0 | 24.0 |
| Polyvinylpyrrolidone (Povidone K25) | 2.0 | 12.0 |
| Sodium Lauryl Sulfate | 2.0 | 12.0 |
| Magnesium Stearate | 0.5 | 3.0 |
| Purified Water ^{b} | — | — |
| Total | 100.0% | 600.0 |
| Film Coat | 3.0 | 18.0 |
| Opaglos 2, green | | |
| 97W11753 | | |

^{a} If assay is other than 100.0%, adjust the amount of input against mannitol accordingly.
^{b} Used in the process, but does not appear in the final tablet product.

Various modifications of the invention, in addition to those described herein, will be apparent to those skilled in the art from the foregoing description. Such modifications are also intended to fall within the scope of the appended claims. Each reference cited in the present application, including patents, published applications, and journal articles, is hereby incorporated by reference in its entirety.

What is claimed is:

1. An Anhydrate Crystal Form (Form D) of 2-(3-fluoro-4-hydroxyphenyl)-7-vinyl-1,3-benzoxazol-5-ol having an X-ray powder diffraction pattern comprising peaks, in terms of 2θ, at about 6.1°, about 10.6°, and about 16.2°.

2. The crystal form of claim 1, wherein the X-ray powder diffraction pattern further comprises at least one peak, in terms of 2θ, selected from at about 9.6°, about 12.3°, and about 14.6°.

3. The crystal form of claim 1 having an X-ray powder diffraction pattern comprising peaks, in terms of 2θat about 6.1°, about 9.6°, about 10.6°, and about 16.2°.

4. The crystal form of claim 1 having an X-ray powder diffraction pattern comprising peaks, in terms of 2θ, at about 6.1°, about 10.6°, about 12.3°, and about 16.2°.

5. The crystal form of claim 1 having an X-ray powder diffraction pattern comprising peaks, in terms of 2θ, at about 6.1°, about 9.6°, about 10.6°, about 12.3°, about 14.6°, and about 16.2°.

6. The crystal form of claim 1 having an X-ray powder diffraction pattern substantially as shown in FIG. 1.

7. An The Anhydrate Crystal Form (Form D) of 2-(3-fluoro-4-hydroxyphenyl)-7-vinyl-1,3-benzoxazol-5-ol having a differential scanning calorimetry trace comprising a melting endotherm having an onset at about 241° C.

8. The crystal form of claim 7 wherein the differential scanning calorimetry trace further comprises an exotherm having an onset at about 102° C.

9. The crystal form of claim 7 having a differential scanning calorimetry trace substantially as shown in FIG. 2.

10. An The Anhydrate Crystal Form (Form D) of 2-(3-fluoro-4-hydroxyphenyl)-7-vinyl-1,3-benzoxazol-5-ol having having a thermogravimetric analysis profile showing less than about 1% weight loss from about 60 to about 150° C.

11. The crystal form of claim 10 having a thermogravimetric analysis profile substantially as shown in FIG. 3.

12. A composition comprising the crystal form of claim 1.

13. The composition of claim 12 wherein said crystal form constitutes at least about 50% by weight of said composition.

14. The composition of claim 12 wherein said crystal form constitutes at least about 80% by weight of said composition.

15. The composition of claim 12 wherein said crystal form constitutes at least about 90% by weight of said composition.

16. The composition of claim 12 wherein said crystal form constitutes at least about 95% by weight of said composition.

17. The composition of claim 12 wherein said crystal form constitutes at least about 98% by weight of said composition.

18. The composition of claim 12 wherein said crystal form constitutes at least about 99% by weight of said composition.

19. The composition of claim 12 wherein said crystal form constitutes at least about 99.5% by weight of said composition.

20. The composition of claim 12 wherein said crystal form constitutes at least about 99.9% by weight of said composition.

21. A composition comprising the crystal form of claim 1 and a pharmaceutically acceptable carrier.

22. A process for preparing the crystal form of claim 1 comprising (a) precipitating a solid from a solution which comprises 2-(3-fluoro-4-hydroxyphenyl)-7-vinyl-1,3-benzoxazol-5-ol and an organic solvent; and (b) drying the solid.

23. The process of claim 22 wherein said organic solvent is a polar organic solvent.

24. The process of claim 23 wherein said organic solvent is a water-miscible organic solvent.

25. The process of claim 22 wherein said organic solvent is selected from an alcohol, a ketone and an organic nitrile.

26. The process of claim 22 wherein said organic solvent is selected from an alcohol and a ketone.

27. The process of claim 22 wherein said organic solvent is selected from methanol, ethanol, isopropanol, acetone and acetonitrile.

28. The process of claim 22 wherein said organic solvent is selected from methanol, ethanol, isopropanol and acetone.

29. The process of claim 22 wherein said solution is substantially free of water.

30. The process of claim 22 wherein said solution comprises less than about 2% by volume of water.

31. The process of claim 22 wherein said solution comprises less than about 1% by volume of water.

32. The process of claim 22 wherein said solution comprises less than about 0.5% by volume of water.

33. The process of claim 22 wherein said precipitating is induced by fast addition of antisolvent to said solution.

34. The process of claim 33 wherein said antisolvent comprises water.

35. The process of claim 33 wherein said antisolvent is water.

36. The process of claim 33 wherein the volume ratio of said antisolvent to said solution is from about 2 to about 3.

37. The process of claim 33 wherein the fast addition is carried out in less than about 15 minutes.

38. The process of claim 33 wherein the fast addition is carried out in less than about 10 minutes.

39. The process of claim 33 wherein the fast addition is carried out in less than about 5 minutes.

40. An anhydrate crystal form (Form D) of 2-(3-fluoro-4-hydroxyphenyl)-7-vinyl-1,3-benzoxazol-5-ol prepared by the process of claim 22.

41. A method of modulating an estrogen receptor comprising contacting said receptor with the crystal form of claim 1.

42. A method of treating prostatitis, interstitial cystitis, inflammatory bowel disease, Crohn's disease, ulcerative proctitis, colitis, prostatic hypertrophy, uterine leiomyomas, breast cancer, endometrial cancer, polycystic ovary syndrome, endometrial polyps, endometriosis, benign breast disease, adenomyosis, ovarian cancer, melanoma, prostrate cancer, colon cancer, glioma, astioblastomia, free radical induced disease states, vaginal or vulvar atrophy, atrophic vaginitis, vaginal dryness, pruritus, dyspareunia, dysuria, frequent urination, urinary incontinence, urinary tract infections, vasomotor symptoms, arthritis, joint swelling or erosion, joint damage secondary to arthroscopic or surgical procedures, psoriasis, dermatitis, ischemia, reperfusion injury, asthma, pleurisy, multiple sclerosis, systemic lupus erythematosis, uveitis, sepsis, hemmorhagic shock, or type II diabetes, in a mammal in need thereof, which comprises providing to said mammal a therapeutically effective amount of the crystal form of claim 1.

43. A method of lowering cholesterol, triglycerides, Lp(a), or LDL levels; inhibiting or treating hypercholesteremia, hyperlipidemia, cardiovascular disease, atherosclerosis, hypertension, peripheral vascular disease, restenosis, or vasospasm; or inhibiting vascular wall damage from cellular events leading toward immune mediated vascular damage in a mammal in need thereof, which comprises providing to said mammal a therapeutically effective amount of the crystal form of claim 1.

44. A method of providing cognition enhancement or neuroprotection; or treating or inhibiting senile dementias, Alzheimer's disease, cognitive decline, stroke, anxiety, or neurodegenerative disorders in a mammal in need thereof, which comprises providing to said mammal an effective amount of the crystal form of claim 1.

45. A method of inhibiting conception in a mammal in need thereof, which comprises providing to said mammal an effective amount of the crystal form of claim 1.
